(19) United States
(12) Patent Application Publication
(10) Pub. No.: US 2007/0191933 A1
(43) Pub. Date: Aug. 16, 2007

(54) Reduction of restenosis

(76) Inventor: Werner Krause, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/594,992

(22) Filed: Nov. 9, 2006

Related U.S. Application Data
(60) Provisional application No. 60/735,219, filed on Nov. 10, 2005.

(51) Int. Cl. A61F 2/82

(52) U.S. Cl. 623/1.42

(57) ABSTRACT

A method of reducing restenosis comprises administering to a patient a stent and reducing, shutting down or modifying functioning of the immune system in a controlled manner. In a preferred embodiment T-cell depletion or T-cell modification is used for controlling the immune system. The T-cell depletor or T-cell modifier is administered either separately or as part of the stent. Alternatively, an ex-vivo procedure may be used.

### FIELD OF THE INVENTION

The present invention relates to the treatment of patients in preparation of, during or after stent implantation. The invention involves temporarily shutting down or decreasing the function of the body's immune system either locally or in the whole organism in a controlled way. In a preferred embodiment the number or the function of T-cells are temporarily reduced. T-cells may also be depleted completely for a limited period of time. The T-cell reducing/depleting/modifying procedure may be performed, either before, during or after stent placement or—as one or more agents—can be part of the stent. This procedure is able to effectively prevent restenosis.

### BACKGROUND OF THE INVENTION

Atherosclerosis is a condition in which fatty material is deposited along the walls of arteries. This fatty material thickens, hardens, and may eventually block the arteries (http://www.nlm.nih.gov/). Atherosclerosis is a common disorder of the arteries. Fat, cholesterol, and other substances accumulate in the walls of arteries and form “atheromas” or plaques. Eventually, this fatty tissue can erode the wall of the artery, diminish its elasticity and interfere with blood flow. Plaques can also rupture, causing debris to migrate downstream within an artery. This is a common cause of heart attack and stroke.

Clots can also form around the plaque deposits, further interfering with blood flow and posing added danger if they break off and travel to the heart, lungs, or brain. When blood flow in the arteries to heart muscle becomes severely restricted, it leads to symptoms like chest pain. Risk factors include smoking, diabetes, obesity, high blood cholesterol, a diet high in fats, and having a personal or family history of heart disease. Cerebrovascular disease, peripheral vascular disease, high blood pressure, and kidney disease involving dialysis are also disorders that may be associated with atherosclerosis. Atherosclerosis may not be diagnosed until symptoms develop.

To some extent, the body will protect itself by forming new blood vessels around the affected area (collaterals). Medications may be recommended to reduce fats and cholesterol in blood; a low-fat diet, weight loss, and exercise are also usually suggested. Control of high blood pressure is also important. Medications include cholestyramine, colestipol, nicotinic acid, gemfibrozil, probucol, atorvastatin, lovastatin, and others. Aspirin, ticlopidine, and clopidogrel (inhibitors of platelet clumping) or anti-coagulants may be used to reduce the risk of clot formation.

Surgically removing deposits (endarterectomy) may be recommended in some cases. A bypass graft is the most invasive procedure. It uses a normal artery or vein from the patient to create a bridge that bypasses the blocked section of the artery.

Balloon angioplasty uses a balloon-tipped catheter to flatten plaque and increase the blood flow past the deposits. The technique is used to open the arteries of the heart and other arteries in the body. Another widely used technique is stenting, which consists of implanting a small metal device, a stent, inside the artery (usually following angioplasty) to keep the artery open.

A stent is any material that is used to hold tissue in place. A stent is often used to support tissues while healing takes place. A stent can keep tube-shaped structures such as blood vessels or ureters (the tubes that drain the kidney) open after a surgical procedure.

An intraluminal coronary artery stent is a small, self-expanding, metal mesh tube that is placed within a coronary artery to keep the vessel open. It may be used during coronary artery bypass graft surgery to keep the grafted vessel open, after balloon angioplasty to prevent reclosure of the blood vessel, or during other heart surgeries.

In more than 70% of cardiac interventions today, a stent is used, usually following a balloon angioplasty (http://www.ptca.org). Sometimes the stent is used as the initial therapy, called “direct stenting.” There are currently clinical trials being conducted to determine the benefits of direct stenting over balloon-plus-stent.

Even if the stent is utilized as the primary therapy, the process still involves a balloon, for the stent itself is mounted on an angioplasty balloon in order for it to be delivered to the diseased area and deployed. The balloon is inflated, and the stent along with it. When the balloon is deflated and withdrawn, the stent remains in place, serving as a permanent scaffolding for the newly widened artery. Within a few weeks, the natural lining of the artery, called the endothelium, grows over the metallic surface of the stent.

Stents have virtually eliminated many of the complications that used to accompany “plain old balloon angioplasty” (POBA) such as abrupt and unpredictable closure of the vessel which resulted in emergency bypass surgery. The additional structural strength of the stent can also help keep the artery open while the healing process progresses.

The concept of the stent grew directly out of interventional cardiologists' experience with angioplasty balloons in the first decade of use (1977-87). Sometimes the wall of the coronary artery became weakened after balloon dilatation. Although the artery would be opened successfully using a balloon, in a small percentage of cases, the artery would collapse after the balloon was deflated—sometimes this might not happen until the patient had been moved to the recovery room. Since there was no interventional “fix” available, the only option for this patient was emergency bypass graft surgery to repair the problem.

A second problem soon became evident as well. Approximately 30% of all coronary arteries began to close up again after balloon angioplasty. By the mid-80's various radiologists and cardiologists were working on solutions to these problems, designing new devices in hopes they would provide more safety and durability to the procedures. Lasers, tiny “shavers,” rotational “polishers”—many tools were miniaturized to be delivered via catheter.

One such device was the stent, a metal tube or “scaffold” that was inserted after balloon angioplasty. The stent itself was mounted on a balloon and could be opened once inside the coronary artery. Julio Palmaz and Richard Schatz were working on such a stent in the United States; others in Europe were developing their own designs. In 1986, working in Toulouse, France, Jacques Puel and Ulrich Sigwart inserted the first stent into a human coronary artery. In 1994 the first Palmaz-Schatz stent was approved for use in the United States. Over the next decade, several generations of bare metal stents were developed, with each succeeding one being more flexible and easier to deliver to the narrowing.

But while stents virtually eliminated many of the complications of abrupt artery closure, restenosis persisted. Although the rates were somewhat lower, bare metal stents still experienced reblocking (typically at six-months) in about 25% of cases, necessitating a repeat procedure. The interventional cardiology community also learned that restenosis, rather than being a recurrence of coronary artery disease, actually was the body's response to what Andreas Gruentzig called the “controlled injury” of angioplasty and was characterized by growth of smooth muscle cells—roughly analogous to a scar forming over an injury.

Experimental and clinical data indicate that leukocytes may be central to intimal growth after mechanical arterial injury (Costa et al., Circulation 2005; 111: 2257-2273) such as balloon angioplasty and stent deployment. In animal models of vascular injury, leukocytes are recruited as a precursor to intimal thickening. In animal models in which a stent is deployed to produce deep vessel wall trauma, a brisk early inflammatory response is induced with abundant surface-adherent neutrophils and monocytes. Days and weeks later, macrophages accumulate within the developing neointima and are observed clustering around stent struts. The number of vessel wall monocytes/macrophages is positively correlated with the neointimal area, suggesting a possible causal role for monocytes in restenosis. Costa and others have shown that blockade of early monocyte recruitment results in reduced late neointimal thickening. Leukocytes likely modulate vascular repair through multiple mechanisms. Inflammatory cells may contribute to neointimal thickening because of their direct bulk within the intima, generation of injurious reactive oxygen intermediates, elaboration of growth and chemotactic factors, or production of enzymes (e.g. matrix metalloproteins, cathepsin S) capable of degrading extracellular constitutents and thereby facilitating cell migration.

Systemic markers of inflammation also appear to be predictive of restenosis after balloon angioplasty. Stenting of patients with stable angina and low C-reactive protein levels at baseline is associated with a transient rise in C-reactive protein that returns to baseline within 48 to 72 hours. Sustained elevations of C-reactive protein are associated with an increased risk of clinical and angiographic restenosis. Using flow cytometry, several groups have reported independently that balloon angioplasty and stenting are associated with upregulation of neutrophil CD11b that is positively correlated with clinical restenosis and late lumen loss and that cell activation occurred across the mechanically injured vessel.

More and more, the solution moved away from the purely mechanical devices of the 90's and toward pharmacologic advances that were being made. If interventional medicine, using the body's circulatory system as a “highway” to deliver therapy, worked with devices, it could also work with medicines. Physicians and companies began testing a variety of drugs that were known to interrupt the biological processes that caused restenosis. Stents were coated with these drugs, sometimes imbedded in a thin polymer for time-release, and clinical trials were begun.

Sometimes referred to as a “coated” or “medicated” stent, a drug-eluting stent is a normal metal stent that has been coated with a pharmacologic agent (drug) that is known to interfere with the process of restenosis (reblocking). Restenosis has a number of causes; it is a very complex process and the solution to its prevention is equally complex. However, in the data gathered so far, the drug-eluting stent has been extremely successful in reducing restenosis from the 20-30% range to single digits. There are three major components to a drug-eluting stent:

Type of stent that carries the drug coating

Method by which the drug is delivered (eluted) by the coating to the arterial wall (polymeric or other)

The drug itself—how does it act in the body to prevent restenosis?

In addition, there are several decisions made by the interventional cardiologist that result in a successful placement:

Correct sizing of the stent length to match the length of the lesion, or blocked area

Correct sizing of the stent diameter to match the thickness of the healthy part of the artery

Sufficient deployment of the stent, making sure that the stent, once placed at the optimum site in the blocked artery, is expanded fully to the arterial wall—under-expansion can result in small gaps between the stent and arterial wall which can lead to serious problems such as blood clots, or Sub-Acute Thrombosis (SAT)

Usually the sizing and the assessments of expansion are made by viewing the real-time angiogram in the cath lab, although some cardiologists also are using more detailed information obtained through intravascular ultrasound imaging.

Finally, in addition to aspirin, the patient must take an anti-clotting drug, such as clopidogrel or ticlopidine (brand names Plavix and Ticlid) for up to six months after the stenting, to prevent the blood from reacting to the new device by thickening and clogging up the newly expanded artery (thrombosis). Ideally a smooth, thin layer of endothelial cells (the inner lining of the blood vessel) grows over the stent during this period and the device is incorporated into the artery, reducing the tendency for clotting.

Currently two drug-eluting stents, the Cordis CYPHER™ sirolimus-eluting stent and the Boston Scientific TAXUS™ paclitaxel-eluting stent system, have received FDA approval for sale in the United States (the Cypher stent in April 2003; the Taxus stent was approved in March 2004) as well as the CE mark for sale in Europe. In addition, the Cook V-Flex Plus is available in Europe. Medtronic and Guidant both have drug-eluting stent programs in the early stages of clinical trials and are looking to 2005 or 2006 for possible approval.

Both the TAXUS and CYPHER stents have shown significant reduction of restenosis in clinical trials and in the field as well. In October 2003, the FDA issued a warning regarding cases of sub-acute thrombosis (blood clotting) with the CYPHER stent that resulted in some deaths. Upon further study, it seemed that the incidence of thrombosis is no greater than that with bare metal stents. The TAXUS stent uses a different drug coating—while more data is being collected, it seems from the preliminary results that the TAXUS stent may have properties that are beneficial to treating diabetic patients as well.

However, there still remains a significant risk of complications following stent implantation.

### SUMMARY OF THE INVENTION

This invention involves shutting down or “dimming” the immune system—for a certain period of time—in a controlled manner in order to prevent restenosis. This can be done by—for example—reducing or eliminating T-cells in the organism or by reducing their functionality. An advantage of the proposed regimen is that the immune system is not damaged but only shut down or reduced in its function and that this effect is reversible. As soon as the stent has built an endothelium of its own, the number/function of T-cells is allowed to return to normal. After discontinuation of treatment, the immune system becomes fully functional again. However, it will take some time for the normal number of T-cells to reappear. This time depends on the specific drug used for T-cell depletion and on the additional use of immune stimulators such as G-CSF or GM-CSF. The re-establishment of a functioning immune system is not restricted to these two examples (G-CSF or GM-CSF). Any other measures known in the art may be used. During the time of treatment and during the time period of recovery of the immune system, the patients are carefully monitored and treated with anti-bacterial and antiviral drugs in order to prevent viral infections. This prophylaxis is well known to those skilled in the art and constitutes daily life in the treatment of cancer or transplant patients with T-cell depletors (Semin Hematol. 2004 July; 41(3): 224-33, Leuk Lymphoma 2004 April; 45(4): 711-4).

This invention relates to a method of preventing restenosis comprising shutting down or reducing the functionality of the immune system either locally at the site of stent implantation or in the whole organism. This can be done for example by administering to a patient a drug that is able to reduce the number of T-cells or to eliminate them completely or to modify their function. However, any other method of shutting down the immune system or reducing its function may also be utilized.

According to the invention, patients designated for stent implantation or having received a stent are treated with drugs that are able to reduce or kill T-cells or to modify the function of T-cells. As an alternative, the T-cell depletor/modifier may be part of the stent itself and is either presented on its surface or otherwise released by the stent. Drugs of this kind are for example monoclonal antibodies that bind to specific epitopes on T-cells and effectively kill these cells, such as the CD3 or CD4 antigen. A drug binding to the T3 antigen is muromonab-CD3 (Orthoclone OKT3). Another potential epitope is the CD52 antigen, which is found on B-cells and T-cells. An example for an antibody binding to the CD52 epitope is alemtuzumab (Campath). However, the invention is not restricted to these types of compounds. Any T-cell depletor/modifier can be used. Also, any epitope on T-cells to which an antibody can be directed, can be utilized, as can any drug that kills T-cells or reduces their number. Moreover, any other type of drug that is able to kill T-cells or reduces their number or functioning, i.e. any T-cell depletor or T-cell function modifier, irrespective of their individual mechanisms of action, may be used. Another example for a T-cell depletor is anti-thymocyte globulin, ATG (Thymoglobulin). Thymoglobulin is anti-thymocyte rabbit immunoglobulin that induces immunosuppression as a result of T-cell depletion and immune modulation. Thymoglobulin is made up of a variety of antibodies that recognize key receptors on T-cells and leads to inactivation and killing of the T-cells. Regarding drugs, which modify T-cells, all will be appropriate as long as the result is that the T-cells are either reduced or eliminated or their function is affected. One such exemplary modification is an antibody binding to receptors such as those described above or others, where the binding does not kill T-cells, but does modify its function.

T-cell depletion has been extensively demonstrated for drugs like alemtuzumab or Thymoglobulin. A single dose of alemtuzubmab (Campath) is able to kill all circulating T-cells. This is illustrated in FIG. 1 (Weinblatt et al. Arth & Rheum 38(11):1589-1594, 1995). As can be seen from FIG. 1, full recovery of T-cells takes 3 months or longer. If the treatment is repeated, T-cell count will remain at low levels or zero during a prolonged period of time. Alemtuzumab is dosed in CLL three times a week at 30 mg for a total of 4-12 consecutive weeks. The final dose of 30 mg is reached after stepwise increases from 3 mg via 10 mg to 30 mg in the first week. In stenting procedures, much smaller doses will be indicated since the tumor load in CLL takes up most of the drug during administration in the first part of the therapy E.g., asin multiple sclerosis (MS), where alemtuzumab is also studied, and dosing is restricted to five daily doses of 10-30 mg for one week. In MS, the therapy might be repeated after a full year.

T-cell depletion after Thymoglobulin is illustrated in FIG. 2 (taken from the Thymoglobulin Prescribing Information). Thymoglobulin is infused in GVHD prevention intravenously over four to six hours. Typical doses are in the range of 1.5-3.75 mg/kg. Infusions continue daily for one to two weeks. The drug remains active, targeting immune cells for days to weeks after treatment. This schedule is routinely adaptable for use in stent implantation.

T-cell depletion for reducing restenosis per this invention is not restricted to the drugs explicitly mentioned herein. Any drug or method that is able to remove, kill or modify T-cells as described herein may be used. Further examples are described for example in Van Oosterhout et al, Blood 2000, 95: 3693-3701. Alternatively, “tetrameric complexes” may be used or ex-vivo T-cell depletion such as immunomagnetic separation can be used (Y. Xiong, The 2005 Annual Meeting, Cincinnati, Ohio). Other examples include FN18-CRM9, SBA-ER (O'Reilly, Blood 1998; Aversa, JCO 1999), CFE (de Witte, BMT 2000) or leukapheresis using the CliniMACS system. Other physical ex-vivo methods include density gradient fractionation, soybean lectin agglutination+E-rosette depletion, or counterflow centrifugal elutriation. Immunological methods in addition to the ones described above include monoclonal antibodies directed against different receptors on T-cells such as CD6 or CD8: immunotoxins such as anti-CD5-ricin may also be employed.

As can be seen, the T-cell depletors and modifiers can be used according to the invention in amounts and in administration regimens routinely determinable and analogous to known uses of such agents for other purposes. Preferably, the extent of depletion or loss of function of the T-cells is at least about 50%, 60%, 70%, 80%, 90%, and also essentially total elimination.

The treatment described above, consisting of T-cell depletion or modification is either adminstered once or until complete covering of the stent with endothelium has been reached. Thereafter, the immune system is allowed to recover. Since the system had been shut down in a controlled manner, any T-cells that are newly formed will be fully functional. Recovery of the immune system might be supported by drugs known in the art for this purpose. Examples are G-CSF or GM-CSF. However, any other applicable drugs or measures might as well be utilized.

### BRIEF DESCRIPTION OF DRAWINGS

FIG. 1 shows Campath killing T-cells; and

FIG. 2 shows Thymoglobulin depleting T-Cells.

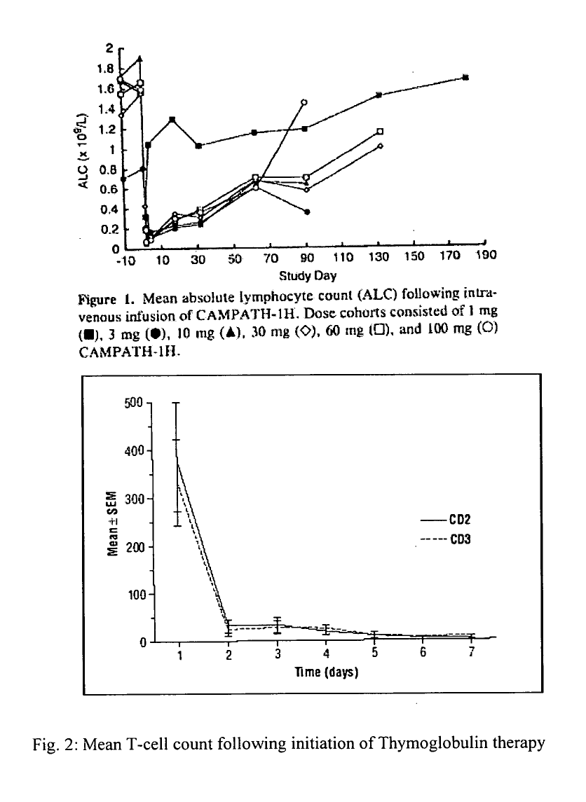

Without further elaboration, it is believed that one skilled in the art can, using the preceding description, utilize the present invention to its fullest extent. The preceding preferred specific embodiments are, therefore, to be construed as merely-illustrative, and not limitative of the remainder of the disclosure in any way whatsoever.

The entire disclosure of the applications, patents and publications, cited herein are incorporated by reference herein.

### EXAMPLES

### Example 1

A study is performed in analogy to the PROVIDENCE (Prevention of Restenosis with Oral Rosiglitazone and the Vision Stent in Diabetics with de novo Coronary Lesions) trial

Patient population: Coronary artery disease

Study Type: Interventional (Percutaneous Coronary Intervention (PCI))

Study Design: Prevention, Randomized, Double-Blind, Placebo Control, Single Group Assignment, Efficacy Study

Primary Outcomes: In-stent and In-segment late lumen loss

Secondary Outcomes: In-stent mean percent diameter stenosis (% DS) and binary restenosis as measured by QCA at post-procedure and at 8 months; TLR and TVR at 30 days, and 8 months post procedure; TVF defined as cardiac death, MI, or TVR at 30 days, 8 months and 1 year post-procedure; Composite of Major Adverse Cardiac Events (MACE); The association of metabolic factors and inflammatory indices including glycemia (HgbA1C), diabetic therapy other than TZDs, HSCRP, coagulation (PAI-1, FIB) and inflammatory marker levels (ADI, MPO, & MMP-9) with the risk for restenosis; Target HgbA1C≦7 for all patients enrolled; Coronary artery stenosis progression in at least one non-stented lesion; Coronary artery stenosis regression in at least one non-stented lesion; Culprit (i.e. stented artery) artery stenosis progression/regression by intravascular ultrasound (IVUS).

Expected Total Enrollment: 100

Eligibility

Ages Eligible for Study: 18 Years and above, Genders Eligible for Study: Both

Inclusion Criteria:

The patients must be >18 years of age;

Patients must be previously diagnosed with type 2 diabetes with documented treatment with insulin, oral hypoglycemics, or diet controlled by medical history. (Undocumented or newly diagnosed diabetics must fulfill the American Diabetes Association Criteria-Report of the Expert Committee on the Diagnosis and Classification of Diabetes Mellitus (Diabetes Care 2003;26:S5-20)).

Diagnosis of angina pectoris defined by Canadian Cardiovascular Society Classification (CCS I, II, III, IV) OR unstable angina pectoris (Braunwald Classification B&C, I-II-III) OR patients with documented silent ischemia;

Treatment of lesions in native coronary arteries requiring stenting. A total of two separate lesions can be stented, located either in the same vessel (at least 10 mm or 1 cm apart) or in two separate vessels. Additional stents may be used for procedural complications such as dissections.

Patient is willing to comply with the specified follow-up evaluation;

Patient provides written informed consent prior to the procedure using a form that is approved by the local Institutional Review Board.

Target lesion is ≧2.0 mm to ≦3.5 mm in diameter (visual estimate);

Individual lesions are ≦25 mm in length located in a native coronary artery;

Target lesions are de novo lesions in native coronary vessels;

Target lesion stenosis is ≧50% and <100% (visual estimate)

Exclusion Criteria:

Patient has experienced an ST-segment elevation myocardial infarction within the preceding 24 hours.

Ejection fraction ≦40%; class III-IV CHF

Active liver disease (ALT >2.5 times upper limit of normal)

Woman of child-bearing potential unless demonstrated 1) negative pregnancy test and 2) clear intention of an accepted method of contraception for eight months after enrollment

Totally occluded vessel (TIMI 0 grade flow);

Impaired renal function (creatinine ≧2.5 mg/dL);

Target lesion involves bifurcation including a side branch ≧2.5 mm in diameter (either stenosis of both main vessel and major branch or stenosis of just major branch) that would require side branch stenting which is likely to occur if side branch is diseased and intended to be stented;

Previous brachytherapy of target vessel;

Recipient of heart transplant;

Patient with a life expectancy less than 12 months;

Known allergies to cobalt, chromium, nickel, aspirin, clopidogrel bisulfate (Plavix®) and/or ticlopidine (Ticlid®), heparin, and/or rosiglitazone (Avandia®), that cannot be medically managed;

Any significant medical condition which in the investigator's opinion may interfere with the patient's optimal participation in the study;

Currently participating in an investigational drug or another device study;

Any contraindication to glycoprotein IIb/IIIa inhibitor therapy;

Current use of any TZD, i.e. rosiglitazone (Avandia®) or pioglitazone (Actos(®)

Chronic or relapse/remitting hemolytic condition

Unprotected left main coronary disease with >50% stenosis;

Patients admitted for treatment of diabetic ketdacidosis >2 times in the past six months (brittle diabetics) and/or the suspicion of type I diabetes;

Target lesion is in a saphenous venous graft or internal mammary graft;

Target lesion is due to restenosis

3 vessel coronary artery disease defined as ≧70% ischemia producing lesions in 3 different epicardial coronary arteries all requiring revascularization (i.e. main left main equivalent)

One day prior to stent implantation, Campath is administered intravenously. A single dose of Campath is infused over 2 hours. Five groups of 20 patients each either receive 0, 1, 5, 10 or 30 mg Campath. Prophylaxis of immediate and late adverse reactions is performed as described in the Campath SmPC for the treatment of CLL patients.

### Example 2

A study is performed as described under Example 1. However, dosing is modified such that more than one dose is administered. The first dose remains prior to stent implantation, subsequent doses are given as soon as lymphocyte counts have reached 75% of baseline levels.

### Example 3

In this example, Campath is part of the stent. The stent is a drug-eluting stent, as known in the art, releasing Campath into the blood stream. To those skilled in the art, it is well known how to produce a drug-eluting stent. Examples are described in US2002032477, US2003108588, EP1362603, U.S. Pat. No. 6,702,850, US2002091433, US2004254638, WO2005007035, which are entirely incorporated by reference herein.

### Example 4

In this example, Campath is fixed at the surface of the stent retaining its full activity. To those skilled in the art, it is well known how to produce a stent with an antibody attached to it. Examples are described in US2005043787, US2004219147, WO03065881, US2003229393, US2002006401, WO0018336, and GB2352635, which are entirely incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of reducing restenosis comprising administering to a patient a stent and temporarily shutting down or reducing the functionality of the immune system either locally or in the whole organism.

2. A method of reducing restenosis comprising administering to a patient a stent and depleting the T-cells of said patient or modiying their functionality.

3. The method of claim 2, wherein T-cell depletion or modification is performed ex vivo.

4. A method of reducing restenosis comprising administering to a patient a stent and a T-cell depletor or a T-cell modifier that reduces the functionality of T-cells.

5. The method of claim 2, wherein the T-cell depletor or modifier is administered to the patient independently of the stent procedure.

6. The method of claim 4, wherein the T-cell depletor or modifier is part of the stent.

7. The method of claim 4, wherein the T-cell depletor or modifier is released from the stent.

8. The method of claim 4, wherein the T-cell depletor or modifier is fixed at the surface of the stent.

9. The method of claim 4, comprising administering a monoclonal antibody directed against CD3.

10. The method of claim 4, comprising administering a monoclonal antibody directed against CD4.

11. The method of claim 4, comprising administering a monoclonal antibody directed against CD52.

12. The method of claim 4, comprising administering muromonab-CD3.

13. The method of claim 4, comprising administering alemtuzumab.

14. The method of claim 4, comprising administering anti-thymocyte globulin.

15. The method of claim 4, comprising T-cell suicide gene transduction (Tk-gene).

16. The method of claim 2, wherein said T-cell depletor or T-cell modifier is administered prior to stent implantation.

17. The method of claim 2, wherein said T-cell depletor or T-cell modifier is administered until endothelialization of the stent has taken place.

18. The method of claim 1, wherein T-cell depletion/modification is accompanied or followed by a treatment for strengthening of the immune system.

19. claim 1 one of the previous claims, further comprising administering of said T-cell depletor or modifier in combination with or followed by G-CSF or GM-CSF treatment.

20. The method of claim 3, wherein a T-cell depletor is administered.

21. The method of claim 1, wherein the T-cell depletor essentially eliminates T-cells.

22. The method of claim 3, wherein a T-cell modulator is administered.

23. The method of claim 1, wherein the T-cell modulator essentially eliminates T-cells.

24. The method of claim 2, for preventing restenosis.

25. The method of claim 1 wherein the extent of T-cell depletion is at least 50%.

26. The method of claim 20 wherein the extent of T-cell function loss is at least 50%.
